(19) United States
(12) Patent Application Publication
(10) Pub. No.: US 2008/0227100 A1
(43) Pub. Date: Sep. 18, 2008

(54) METHOD FOR ESTIMATING hERG INHIBITION OF DRUG CANDIDATES USING MULTIVARIATE PROPERTY AND PHARMACOPHORE SAR

(75) Inventor: Stephen Roger Johnson, Erdenheim, PA (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(73) Assignee: Bristol-Myers Squibb Company

(21) Appl. No.: 12/046,493

(22) Filed: Mar. 12, 2008

Related U.S. Application Data
(60) Provisional application No. 60/894,572, filed on Mar. 13, 2007.

(51) Int. Cl. C12Q 1/68

(52) U.S. Cl. 435/6

(57) ABSTRACT

The present invention provides a computational model and methods of use thereof for predicting whether a compound is likely to inhibit K⁺ flow through the hERG ion channel. Methods for in silico screening of compounds that have a lower likelihood of inhibiting hERG are also provided.

### FIELD OF THE INVENTION

The present invention provides a computational model and methods of use thereof for predicting whether a compound is likely to inhibit K⁺ flow through the hERG ion channel. Methods for in silico screening of compounds that have a lower likelihood of inhibiting hERG are also provided.

### BACKGROUND OF THE INVENTION

Pharmacological blockade of I_{Kr}, the cardiac potassium ion current encoded by the human ether-a-go-go related gene (hERG), has been linked to a delayed membrane potential repolarization, prolonged action potential duration and increased QT interval on the ECG.¹⁻³ This increase in the QT interval is a major risk factor for Torsades de pointes,⁴ a serious cardiac arrhythmia occasionally resulting in death. Potent blockade of the hERG channel is now a primary concern in the drug discovery process, resulting in a correspondingly significant chemistry effort directed toward minimizing this undesirable activity.

The growth in the literature regarding how potential drugs block the hERG channel is testimony to the importance of this liability in the drug discovery process. The primary efforts are the hERG alanine-scanning experiments conducted by Sanguinetti and coworkers⁵ that identified the aromatic residues F656 and Y652 as playing a significant role in drug-induced blockade. Of the compounds studied thus far, only some lower potency hERG inhibitors have not been highly affected by mutation of Y652.⁶⁷ Still more recently, additional mutagenesis studies have shown the importance of Pi-Cation interactions between ligands and the Y652 of hERG,⁸ as well as the role of Y652 in voltage-dependent inhibition.^{7,9,10}

The number of reviews¹¹⁻¹⁷ and primary reports^{8,11,12,15,18-35} of computational models of hERG inhibition continues to grow. A particularly notable review was made by Jamieson³⁶ et al., who discuss practical avenues (including modeling) for mitigating hERG inhibition. For the most part, there appears to be a general consensus on the primary drivers of hERG binding, namely Pi-cation interactions and usually some combination of Pi-Pi and/or hydrophobic interactions with F656. Still, the diversity of potent inhibitors and the broad range of activity for compounds with typical hERG pharmacophores continue to be a vexing problem without a firmly established path for optimization.

The desire for a computational model for predicting hERG inhibition is two-fold. First, the broad SAR (structure activity relationship) demonstrated by the array of potent hERG blockers has often frustrated medicinal chemistry efforts to work around this critical liability. Thus, there exists a critical need in the art for a tool that can provide guidance to balance against target potency models and SAR in an effort to speed discovery programs. Second, the most trusted assay technology for assessing hERG blockade, patch-clamp electrophysiology, is very labor intensive and struggles to keep pace with the number of compounds being considered an increasingly high-throughput environment. Thus, there also exists in the art a need for an in silico tool to complement other technologies that aim to address this throughput dilemma such as FLIPR assays,³⁷ radioligand binding assays,³⁸ and more recently, automated patch-clamp instruments.³⁹ Accordingly, there exists in the art a need for an in silico tool to prioritize compounds for assay analysis to assess which compounds are least likely to inhibit hERG activity. Such a tool would greatly impact the costs of toxicity screening in discovery research. The present invention presents such an in silico model for predicting the likelihood that a compound will inhibit hERG that has advantages over those models known in the art.

### BRIEF SUMMARY OF THE INVENTION

A method of predicting the likelihood that a compound will inhibit the potassium ion current activity of the human ether-a-go-go gene (hERG), comprising the step of determining whether said compound comprises one or more of the descriptors selected from the group consisting of: a.) the descriptor according to Formula (I); b.) the descriptor according to Formula (II); and c.) the descriptor according to Formula (III).

The method of predicting the likelihood that a compound will inhibit the potassium ion current activity of the human ether-a-go-go gene (hERG), wherein said method comprises determining whether said compound comprises both the descriptor according to Formula (I) and the descriptor according to Formula (II).

The method of predicting the likelihood that a compound will inhibit the potassium ion current activity of the human ether-a-go-go gene (hERG), wherein a compound comprising both the descriptor according to Formula (I) and the descriptor according to Formula (II) has a lower likelihood of inhibiting the potassium ion current activity of the human ether-a-go-go gene (hERG) relative to a compound having only the descriptor according to Formula (I).

The method of predicting the likelihood that a compound will inhibit the potassium ion current activity of the human ether-a-go-go gene (hERG), wherein method said comprises determining whether said compound comprises the descriptor according to Formula (IlI).

The method of predicting the likelihood that a compound will inhibit the potassium ion current activity of the human ether-a-go-go gene (hERG), wherein a compound comprising the descriptor according to Formula (III) has a higher likelihood of inhibiting the potassium ion current activity of the human ether-a-go-go gene (hERG) relative to a compound lacking this descriptor.

The method of predicting the likelihood that a compound will inhibit the potassium ion current activity of the human ether-a-go-go gene (hERG), wherein method said comprises determining whether said compound comprises the descriptor according to Formula (I), and further comprises determining whether said compound comprises an aromatic ring with sufficient electrostatic potential to enable Pi-stacking.

The method of predicting the likelihood that a compound will inhibit the potassium ion current activity of the human ether-a-go-go gene (hERG), wherein a compound comprising the descriptor according to Formula (I) in conjunction with an aromatic ring capable of Pi-stacking, has a higher likelihood of inhibiting the potassium ion current activity of the human ether-a-go-go gene (hERG) relative to a compound having only the Formula (I) descriptor.

The method of predicting the likelihood that a compound will inhibit the potassium ion current activity of the human ether-a-go-go gene (hERG), wherein a compound comprising the descriptor according to Formula (III) has about 3 fold higher likelihood of inhibiting the potassium ion current activity of the human ether-a-go-go gene (hERG) relative to a compound lacking this descriptor.

The method predicting the likelihood that a compound will inhibit the potassium ion current activity of the human ether-a-go-go gene (hERG), wherein method said comprises determining whether said compound comprises the descriptor according to Formula (I), and further comprises determining whether said compound comprises an aromatic ring with sufficient electrostatic potential to enable Pi-stacking wherein said aromatic ring is substituted with at least one electron withdrawing group, wherein said compound has about a 5 to about a 30 fold higher likelihood of inhibiting the potassium ion current activity of the human ether-a-go-go gene (hERG) relative to a compound containing an unsubstituted aromatic ring.

The method predicting the likelihood that a compound will inhibit the potassium ion current activity of the human ether-a-go-go gene (hERG), wherein method said comprises determining whether said compound comprises the descriptor according to Formula (I), and further comprises determining whether said compound comprises an aromatic ring with sufficient electrostatic potential to enable Pi-stacking wherein said aromatic ring is substituted with at least one electron donating group, wherein said compound has about a 2 to about a 5 fold lower likelihood of inhibiting the potassium ion current activity of the human ether-a-go-go gene (hERG) relative to a compound containing an unsubstituted aromatic ring.

### BRIEF DESCRIPTION OF THE FIGURES/DRAWINGS

FIG. 1. (A) Shows the agreement between fully determined IC₅₀s from the hERG patch-clamp assay, and IC₅₀s estimated from single-point %-inhibition data. The r²˜0.83 with an RMS error of 0.27 log units. (B)Residuals of estimation versus %inhibition of the single point data. The line is a 20 compound moving average of the residuals.

FIG. 2. Distribution of the correlation coefficients from a Monte Carlo simulation of the effect of the error introduced by estimating IC₅₀s from the percent inhibition data. The simulation suggest that the upper bound on the in silico model performance is r²˜0.8. More likely, the actual performance must be lower as this simulation ignored other sources of experimental error.

FIG. 3. Calculated versus observed plot for the model defined in Table 1.

FIG. 4. Prediction results on discovery compounds post-model development. R²=0.54, RMSe of Prediction=0.63.

FIG. 5. Cumulative distribution plots for the presence of the BR4 and AAL334 pharmacophores.

FIG. 6. Predicted versus observed plot for the compounds in Cluster 0 (see Table 3). The circled compounds are discussed in the text.

FIG. 7. Relationship between topological similarity (Daylight Fingerprints) of validation compound clusters and training data and the correlation between observed and predicted IC₅₀s.

### DETAILED DESCRIPTION OF THE INVENTION

The entire model development cycle described herein was comprised of the iterative development of a series of candidate models (hypotheses). Existing compounds in an internal inventory were then selected for testing in a manual patch-clamp assay to challenge particular features. This usually was accomplished by selecting compounds with a narrow range of values for one feature, but with a broad diversity of values for the other features in the model. In some instances) compounds with combinations of descriptor values that were unusual in the pre-existing data were sought for testing. Several features were eliminated in this process when the coefficient for the feature derived for the new data was not significant or if the coefficient was substantially different from the original hypothesis. At this point, a new hypothesis was generated, and the process repeated. The present invention represents the outcome of this model development process.

TABLE 1
Descriptors and coefficients used in the prediction of hERG IC₅₀s

| Description | Label | Coefficient |
|---|---|---|
| Log D at pH = 6.5 | LogD_{6.5} | −1.576 |
| Dry probe interaction volume at | Vol_D7 | −1.236 |
| −1.4 kcal/mol | | |
| Log P(Octanol-Methylformamide) | LogP_{Oct/NMF} | −2.699 |
| Aromatic ESP Interaction volume @ | aESP, −5 | 1.084 |
| −5 kcal/mol | | |
| Binary interaction pair: Two aromatic | BIP2214 | −0.298 |
| atoms 14 bonds apart | | |
| Binary interaction pair: H-bond acceptor | BIP2611 | −0.317 |
| 11 bonds from an aromatic atom | | |
| DDRR311342 | DDRR | −0.293 |
| BR4 | BR4 | −0.171 |
| AAL334 | AAL | 0.192 |
| Secondary & Tertiary amine indicator | Am | −0.955 |
| Intercept | | 4.744 |

Training Results R²~0.65, RMSe ~0.58
Validation Results R²~0.66, RMSe ~0.47
* Table 2 further elucidates the pharmacophore descriptors

TABLE 2
Definitions of the pharmacophoric descriptors used in the model.

| BR4 | Basic center (B) | Lipophilic (L) |
|---|---|---|
| Basic Center | | 6.0-9.0 |

| AAL334 | H-Acceptor (A) | H-Acceptor (A) | Lipophilic (L) |
|---|---|---|---|
| H-Acceptor | — | 4.0-6.0 | 4.0-6.0 |
| H-Acceptor | — | — | 6.0-9.0 |

| DDRR311342 | H-Donor (D) | H-Donor (D) | Aromatic (R) | Aromatic (R) |
|---|---|---|---|---|
| H-Donor | — | 6.0-9.0 | 2.5-4.0 | 2.5-4.0 |
| H-Donor | — | — | 6.0-9.0 | 9.0-13.0 |
| Aromatic | — | — | — | 4.0-6.0 |

Distances between pharmacophoric points shown in Angstroms.

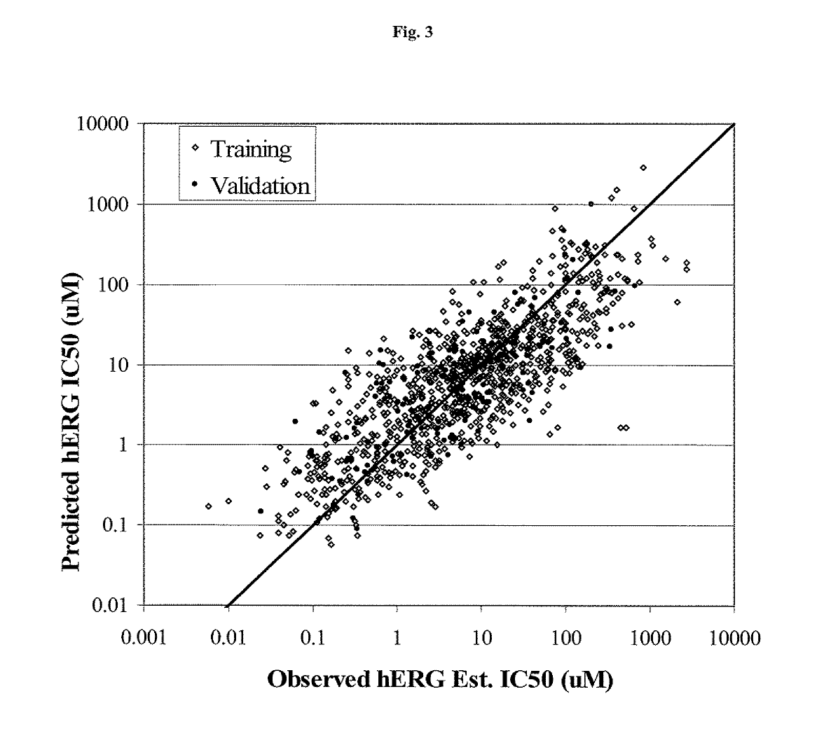

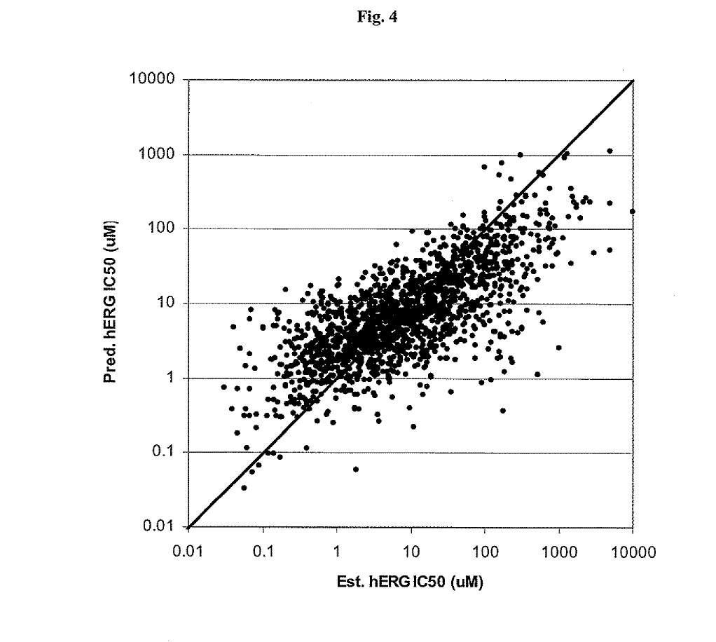

The 10 descriptors and coefficients for the model of the present invention was derived using the approach explained above are shown in Tables 1 and 2. The calculated versus observed plot for the training and test data is shown in FIG. 3. The model is a combination of both pharmacophoric and physicochemical descriptors, several of which are novel compared to the literature. The prediction results for the is 1679 compounds that have been assayed since this model was developed are shown in FIG. 4.

The validation data shown in FIG. 4 are the more interesting tools for analyzing the performance of the model. These compounds were primarily tested in the natural course of discovery programs, although a subset of compounds was tested specifically to challenge the model in weakly populated descriptor space. The model predicts this large data set with an R²=0.54 and an RMSe of prediction of 0.63. Compared to the training and original test data in FIG. 3, there is wide scatter in the prediction plot. In addition, the skew at the extrema of the observed values are more pronounced than seen when the model was developed. Still, the overall prediction is quite satisfactory as a true forward test of the model. More in-depth discussion of this validation data is given below.

A QSAR model and its descriptors therein, like the model of the present invention, serves as the basis for a hypothesis that can be tested to bolster or refute correlations and to establish confidence in the causality of such correlations. The presence of a basic amine has been a feature in many computational models of hERG inhibition. Experimental evidence supports the hypothesis that a basic ionizable center can participate in a Pi-cation interaction with Y652.⁸ The model of the present invention has two such features. The first is a simple indicator variable for a secondary or tertiary basic amine. The second is a pharmacophore consisting of a basic center 6 to 9 Å from the centroid of an aromatic ring. Earlier versions of our model, developed when only a few hundred compounds were available, utilized a pharmacophore very similar to those in the literature.^{23,27,32} Each of these reports utilized basically the same pharmacophores with variations between the use of lipophilic points and aromatic points. However, the statistical power of these pharmacophores has failed to maintain significance with the availability of new compounds from various discovery projects. More recent compounds that contain the prototypical pharmacophore of a base with aromatic and lipophilic groups have had a wider distribution of hERG activity as medicinal chemistry optimization has focused on manipulating the properties of the aromatic systems or basic center that make up the pharmacophore. Additionally newer compounds that lack the distal hydrophobic points have maintained hERG potency. These two changes in trends over time highlight how the reductionist nature of pharmacophores can lead to dramatically different results depending on the particular collection of compounds used to derive them.

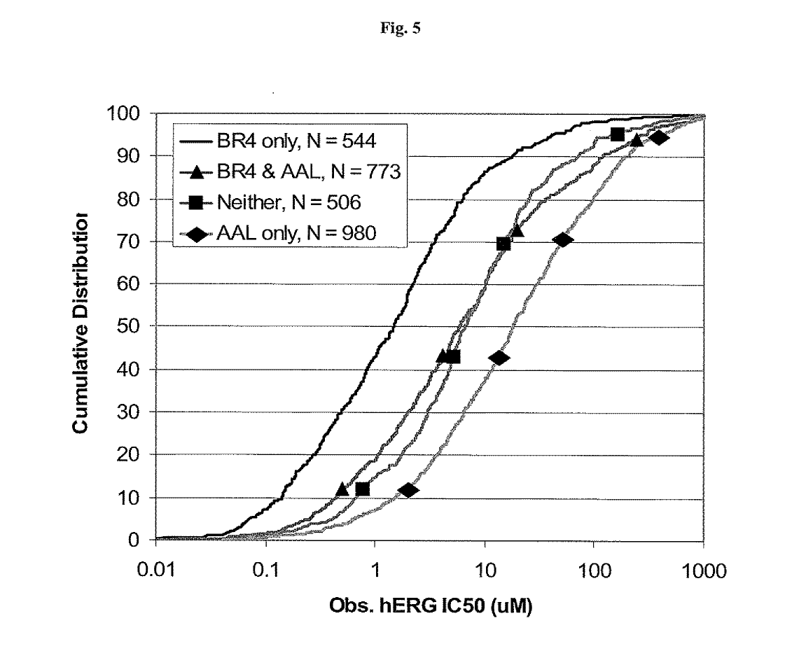

While searching for an explanation for the loss of power of the established hERG binding pharmacophore, a second pharmacophore was identified that seemed to directly mitigate the impact of the BR4 feature. Comprised of two H-bond acceptors and a lipophilic group, the AAL334 pharmacophore chiefly negates the impact of the BR4 pharmacophore when both are present simultaneously. This may prove a viable avenue for the optimization that allows medicinal chemists to retain a basic ionizable center. Additionally, compounds containing AAL334 appear to have slightly lower hERG potency in compounds not containing the BR4 pharmacophore. FIG. 5 shows the cumulative distribution plots of hERG IC₅₀ for compounds with respect to the presence or absence of BR4 and/or AAL334. The activity distribution for compounds with the BR4 pharmacophore but not the AAL334 pharmacophore is shifted toward more potenct IC₅₀s. In contrast, compounds with both pharmacophores have an activity distribution that is nearly identical to those compounds that lack the BR4 pharmacophore. To our knowledge, no similar pharmacophore has appeared in the literature as diminishing hERG activity.

Several additional features in the model highlight the importance of aromatic, lipophilic, and H-bond acceptor groups in hERG potency. While the precision of a 14-bond distance in BIP-6614 between aromatic atoms is difficult to justify, the parameter is identifying multiple aromatic rings that are at opposing ends of a molecule. This is an easily recognizable trait among many potent inhibitors of hERG, with Pi-stacking postulated as an important interaction with both F656 and Y652 in the pore of the channel. A combination of Pi-stacking to these residues coupled with the possibility of H-bond formation is captured in the BIP-2611 feature. Based on crude homology models derived from the KcsA and Kv1.2 structures (not shown), this may be consistent with H-bonds between the ligand and polar residues at the base of the selectivity filter (e.g. Thr623). Such interactions have been hypothesized previously.

Another pharmacophore present in the model is more novel compared to the literature. The feature labeled as DDRR contains two H-bond donors as well as two aromatic rings. Compounds possessing this feature are, on average, 3-fold more potent than compounds that do not contain this pharmacophore. It is actually not a trivial exercise to rationalize this pharmacophore with a homology model of hERG. Few opportunities are apparent for an H-bond donor making interactions with sidechains. It is possible for such interactions to form with Thr623, although the equivalent feature using an H-bond acceptor does not show a propensity towards increased hERG potency.

One feature of the model that arose specifically as a result of a series of compounds from a discovery project was the electrostatic potential (ESP) around aromatic rings contoured at −5 kcal/mol. A number of papers suggesting the importance of electrostatic potential in aromatic Pi-stacking have appeared in the literature.⁵⁶⁻⁵⁹ We observed a trend among a series of compounds in which compounds with electron withdrawing groups around a phenyl ring had a 5-30 fold increase in potency over that of the unsubstituted ring. Compounds with electron donating groups attached to the ring had a 2-5 fold decrease in potency compared to the unsubstituted ring. Based on this and similar trends in our corporate database, several series of compounds were selected for testing, and the trend appeared to be generally consistent. A feature (aESP,-5) was designed specifically to capture aromatic rings that were relatively electron deficient. This feature is now among the most important features in the model based on coefficient, and has already found significant use in internal discovery projects as an avenue for optimization.

Jamison, et al.³⁶ propose the manipulation of the electrostatic around aryl rings as an avenue toward remediation of hERG liabilities. In contrast to our results, they propose the addition of electron withdrawing groups to decrease hERG potency. Perry et al.⁶⁰ discuss the effect of changing the para substituent on a series of clofilium analogs. They observe that the potency is increased with a more polarizable group at the para position. This is more consistent with our findings. A number of the theoretical studies of aromatic Pi-stacking do show that the addition of electron donating groups can make Pi-stacking more favorable. However, these studies are performed relative to benzene, whereas phenol would be a better model system for interactions with Y652.

It has been appreciated for some time that the formal charge on a compound can have great impact on hERG inhibition. In general compounds possessing an acidic group have greatly diminished potency. This comes as little surprise as potassium ion channels are designed to stabilize a monovalent cation in the water filled cavity.⁶¹ Nonetheless, not all acidic compounds are completely inactive against hERG. In our database acidic compounds are as potent as 60% inhibition at 1 μM (est. IC₅₀˜6 μM) with the median potency being 24% inhibition at 30 μM (est. IC₅₀˜95 μM). In contrast, the median potency of basic compounds is 4.7 μM and for neutral compounds the median potency is 50% inhibition at 10 μM (est. IC₅₀˜10 μM). The LogP_{Oct/NMF} feature (octanol-N-methylformamide partition coefficient) captures this trend quite well, as well as identifying compounds with low LogD_{6.5} that still show significant potency. This feature is calculated using the free energies of solvation calculated by OmniSol. In contrast to the LogD_{6.5} feature where the distributions for acidic and basic compounds are nearly identical, values for the LogP_{Oct/NMF} of the acidic compounds are substantially more negative than for the basic compounds. A sensitivity analysis of the correlation of the LogPO_{Oct/NMF} to the hERG IC₅₀ was performed by manipulating the solvent parameters of N-methylformamide used in Omnisol. This analysis showed that the correlation was highly sensitive to the values of Abraham's H-bond acidity parameter,⁶² ρα₂^{H}, and somewhat less sensitive to the H-bond basicity parameter.

The Volsurf feature, D7, captures the impact of hydrophobicity on affinity for the hERG channel. This may be a representation of the amount and character of hydrophobic surface in the ligand that could be buried in accordance with the hydrophobic effect. In any event, increasing lipophilicity is a well-recognized route to increased potency against hERG.³⁶

We have clustered the 1679 validation compounds based on Daylight Fingerprint⁶³ Tanimoto using an average linkage method with a similarity cutoff of 0.7. Generally each cluster contains only a single chemotype, although a chemotype can be split across more than one cluster. This view of the data provides an analysis of how the model would perform within a group of compounds that would be generated within a single discovery project. Table 3 shows the prediction statistics for the 35 largest clusters of compounds, representing approximately 50% of the validation compounds. While the quality of the prediction results vary quite dramatically from cluster to cluster, the results support the use of the model in generating hypotheses of how to approach hERG optimization within many projects. In fact, the model has been used in the optimization efforts for several of the clusters of compounds in Table 3.

TABLE 3
Model performance for clusters of related compounds.

| | | | Spearman | | | Max. Similarity |
|---|---|---|---|---|---|---|
| Cluster | R² | RMSe | Rho | Range^{a} | Num | to Training Data |
| 0 | 0.32 | 0.45 | 0.50 | 3.38 | 84 | 0.40 |
| 1 | 0.37 | 0.49 | 0.65 | 2.14 | 70 | 0.96 |
| 2 | 0.69 | 0.75 | 0.84 | 2.92 | 50 | 0.52 |
| 3 | 0.76 | 0.47 | 0.90 | 3.63 | 47 | 0.46 |
| 4 | 0.44 | 0.64 | 0.60 | 2.62 | 45 | 0.76 |
| 5 | 0.55 | 0.43 | 0.75 | 2.34 | 42 | 0.98 |
| 6 | 0.34 | 0.96 | 0.59 | 2.98 | 38 | 0.64 |
| 7 | 0.50 | 0.42 | 0.62 | 2.28 | 28 | 0.99 |
| 8 | 0.59 | 0.60 | 0.66 | 2.89 | 25 | 0.75 |
| 9 | 0.71 | 0.47 | 0.85 | 2.36 | 24 | 0.98 |
| 10 | 0.71 | 0.53 | 0.83 | 3.14 | 23 | 0.89 |
| 11 | 0.37 | 0.64 | 0.67 | 2.63 | 22 | 0.42 |
| 12 | 0.45 | 0.26 | 0.69 | 1.18 | 19 | 0.49 |
| 13 | 0.46 | 0.38 | 0.68 | 1.46 | 18 | 0.89 |
| 14 | 0.86 | 0.38 | 0.94 | 2.60 | 18 | 0.46 |
| 15 | 0.42 | 0.80 | 0.53 | 1.48 | 17 | 0.95 |
| 16 | 0.45 | 0.50 | 0.70 | 1.89 | 17 | 0.41 |
| 17 | 0.40 | 0.71 | 0.55 | 1.87 | 16 | 0.96 |
| 18 | 0.45 | 1.30 | 0.71 | 2.45 | 15 | 0.42 |
| 19 | 0.42 | 0.40 | 0.47 | 1.64 | 15 | 0.35 |
| 20 | 0.86 | 0.91 | 0.89 | 4.52 | 15 | 0.99 |
| 21 | 0.23 | 0.33 | 0.39 | 1.45 | 14 | 0.40 |
| 22 | 0.72 | 0.71 | 0.72 | 3.15 | 14 | 0.38 |
| 23 | 0.85 | 0.67 | 0.96 | 3.79 | 14 | 0.37 |
| 24 | 0.01 | 0.63 | 0.05 | 2.16 | 14 | 0.41 |
| 25 | 0.49 | 0.35 | 0.60 | 1.72 | 12 | 0.44 |
| 26 | 0.50 | 0.97 | 0.64 | 2.42 | 12 | 0.50 |
| 27 | 0.01 | 0.61 | 0.10 | 0.75 | 12 | 0.34 |
| 28 | 0.61 | 0.54 | 0.64 | 2.66 | 12 | 0.49 |
| 29 | 0.13 | 0.54 | 0.35 | 1.01 | 11 | 0.90 |
| 30 | 0.92 | 0.40 | 0.87 | 1.62 | 10 | 0.91 |
| 31 | 0.19 | 0.78 | 0.37 | 2.41 | 10 | 0.42 |
| 32 | 0.29 | 0.48 | 0.55 | 1.11 | 10 | 0.37 |
| 33 | 0.48 | 0.32 | 0.48 | 1.55 | 10 | 0.33 |
| 34 | 0.52 | 0.62 | 0.72 | 2.24 | 10 | 0.66 |
| Average | 0.49 | 0.58 | 0.63 | | 813 | |
| Median | 0.46 | 0.54 | 0.65 | | | |
| Std. | 0.23 | 0.22 | 0.21 | | | |
| Dev. | | | | | | |

^{a}Range of Log₁₀(hERG IC₅₀) of compounds within each cluster.

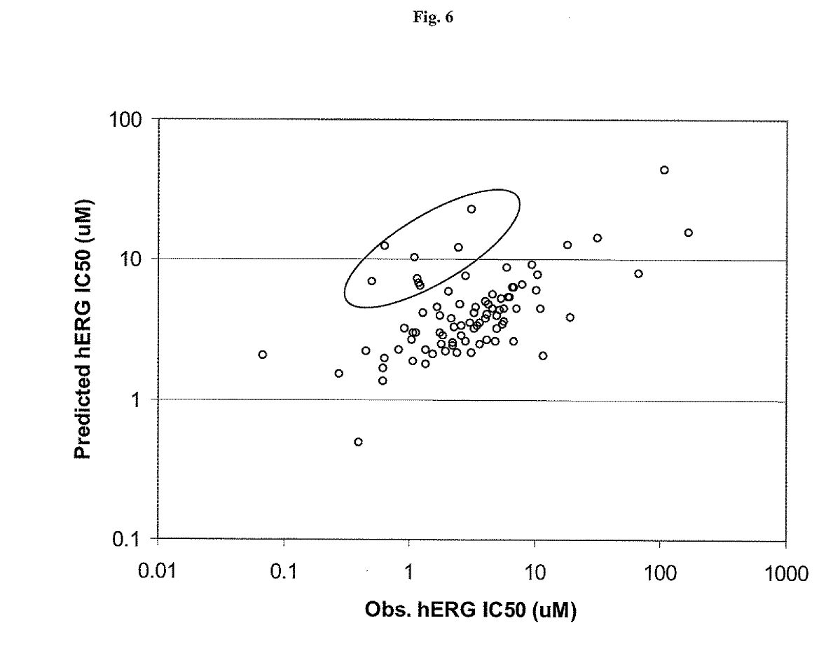

FIG. 6 shows the prediction results for compounds in the largest cluster of compounds, cluster 0. The discovery project responsible for these compounds frequently used the model in the optimization of hERG potency. The graphical results shown in the Figure appear much better than the R² of 0.32 implies. Circled in the Figure are a group of compounds predicted to be substantially less active than the observed data. These compounds all contain either a pyrimidine or a pyrazine in place of a particular phenyl ring present in the well-predicted compounds. This particular error is also observed in other unrelated clusters of compounds. We believe that this is a result of the simplistic nature of the ESP feature used to encode the importance of Pi-stacking interactions. The view of the ESP used here was specifically conceived to capture Pi-stacking interactions for phenyl rings. There is ample evidence for Pi-stacking in other aromatic rings, although the ESP plots for heterocycles discussed in the literature are often quite different from those for a phenyl ring. While the particular quantitative estimate from the model is subject to these errors the underlying concept of modulating the ESP of the ring has played an important role in optimizing hERG liabilities in several projects. Future model refinement will entail a more sophisticated treatment of these interactions.

Also evident from Table 3 is that there is no single statistic that adequately summarizes the utility of a model. For example, cluster 11 has a relatively low R² of 0.37, but an acceptable ability to rank order compounds based on the Spearman's Rho of 0.67. The same is true for cluster 1, although these compounds are much more similar to the training data. The predictions for cluster 20, however, correlate very highly (R²=0.86) with the observed data. However, the RMSe of prediction of 0.91 is substantially higher than the overall RMSe of 0.63. While this is well-known to those skilled in the art of modeling, it is frequently a barrier to the exploitation of the model by discovery projects.

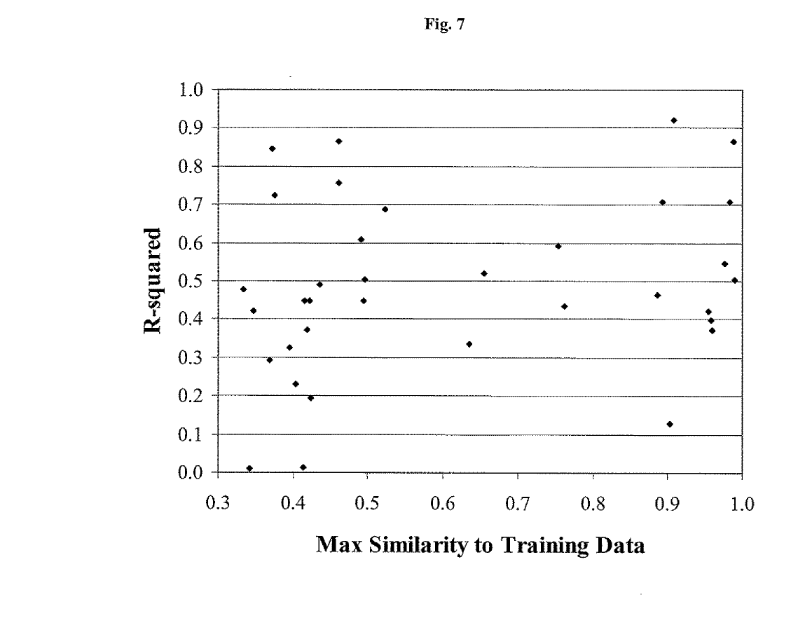

There is debate in the modeling community about how to identify a compound as being similar to the training data, and thus likely to be predicted reliably. Table 3 shows the maximum Daylight fingerprint Tanimoto similarity of any compound in each cluster to a compound in the training data used to generate the model. FIG. 7 shows that there is no correlation between the R² of prediction for the compounds in a cluster, and the similarity of the compounds in that cluster to the training data. In contrast, the overall correlation for the validation set improves to R²=0.72 when considering only those compounds with a Tanimoto similarity greater than 0.65 to a training set member. This disparity arises because many of the clusters with low similarity to the training data have significantly different slopes or constants (intercepts) for the predicted v, observed plot than those of the high similarity clusters even while the correlation coefficient for the cluster may be acceptable.

### Conclusions

The inventors have presented an in silico model of hERG inhibition that is based upon a large diverse collection of discovery compounds. Much of the information used in the model is consistent with previously established literature. However, the model does include a number of factors that have not been considered explicitly previously. The AAL334 pharmacophore largely negates the impact of the common basic amine and aromatic system that is so prevalent among hERG inhibitors (i.e., BR4). Another is the use of electrostatic potentials to assess potential Pi-stacking interactions with Y652 and/or F656. The inclusion of the LogP_{Oct/NMF} improves the prediction over the range of activities seen for acidic and other polar compounds. Each of these features suggests different avenues for medicinal chemistry optimization of hERG affinity.

Further, the ability of the model to predict across an array of different chemistries was demonstrated. While much room for improvement remains, the capability for quality predictions across several different chemotypes allows discovery projects to leverage knowledge from one project to advance another. By analyzing the model performance within clusters of compounds specific weaknesses of the model are much more easily brought to light. This facilitates the revision of the assumptions made during model generation, allowing for a continual improvement and retention of knowledge as it is generated within discovery.

The derivation of a model of this sort requires close analysis of the data throughout the process. Frequently in drug discovery, incomplete data is collected that, while it meets the needs of many users, presents significant challenges for use in modeling.⁶⁴ We presented a simple Monte Carlo method capable of providing insight on the impact of data manipulation and/or data reproducibility. Indeed, all too often in the QSAR literature data is fit to a greater degree than is reasonable given the quality of the data available. Simple methods such as that discussed here can provide guidance as to when a model has reached the degree of precision and accuracy supported by the underlying data.

There are a number of avenues by which the model could be refined and improved. Improvements to the electrostatic potential descriptors used to describe Pi-stacking interactions are needed to better account for heterocycles. It is also likely that different ESP features intended to differentially encode face-to-face or face-to-edge interactions may significantly improve our understanding of hERG inhibition. Other improvements would include coupling the ligand-based model to a structure-based approach to better capture stereochemical differences and steric constraints, as well as more sophisticated pharmacophoric descriptors. All of these are currently areas of active research within our effort.

Descriptors of the Model

The model of the present invention encompasses one or more of the descriptors having the general structure of formula (I), also referred to as the BR4 descriptor; (II), also referred to as the AAL334 descriptor; and/or (III), also referred to as the DDRR311342 descriptor,

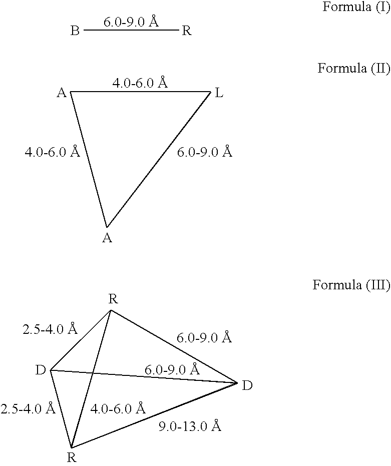

wherein, “A” represents a hydrogen bond acceptor; “R” represents a centroid of an aromatic ring; “L” represents a lipophilic atom; “B” represents a basic ionizable atom; and “D” represents a hydrogen bond donor atom. One skilled in the art of chemistry would appreciate the meaning of each of these terms, and readily be able to identify compounds that contain one or more atoms, moieties, functional groups, and the like, that meet the requirements of each descriptor in terms of both function and space.

Non-limiting examples of a hydrogen bond acceptor include any oxygen, except for those contained within a nitro groups or ethers in which the oxygen is directly attached to an aromatic atom. Additional non-limiting examples a hydrogen bond acceptor are the oxygen atom in carbonyls, esters, hydroxyls, amides, ethers, furan, oxazoles, isoxazoles, oxadiazoles, pyran, dioxane, morpholine, or the like. Further non-limiting examples a hydrogen bond acceptor also include unprotonated nitrogens, except for the nitrogen in nitro groups, amides, anilines, or quaternary nitrogen. An example of a nitrogen H-bond acceptor is the nitrogen in tertiary amines, cyano, imidazole, pyrazole, isoxazole, pyridine, pyrimidine, triazole, or the like. Additional examples are known in the art or otherwise disclosed herein.

Aside from the hydrogen bond acceptor examples provided herein or otherwise known in the art, hydrogen bond acceptors may also be identified using SMARTS patterns with the OEChem Toolkit (v1.4.2, OpenEye Scientific Software, Sante Fe, N. Mex.). An example of the SMARTS patterns used to detect a the presence of a hydrogen bond acceptor are: [O,o;!$(O˜N˜O);!$(O(C)a)] and [n,N;HO,!$(N(˜O)˜O);!v4;!$(NC═O);!$(Nc)]. Alternatively, any substructure search algorithm could be used.

Non-limiting examples of the centroid of an aromatic ring include, phenyl rings or aromatic heterocyclic rings such as pyridine, pyrimidine. The centroid is determined by identifying the geometric center of the atoms comprising the aromatic ring. Additional examples are known in the art or otherwise disclosed herein.

Aside from the centroid of an aromatic ring examples provided herein or otherwise known in the art, centroid of an aromatic ring may also be identified using SMARTS patterns with the OEChem Toolkit (v1.4.2, OpenEye Scientific Software, Sante Fe, N. Mex.). An example of the SMARTS patterns used to detect a the presence of a aromatic ring are to determine the smallest rings containing only atoms that match the SMARTS: [a]. Alternatively, any substructure search algorithm could be used.

Non-limiting examples of the lipophilic atoms include, non-aromatic carbons atoms that are at least two bonds from any heteroatom, and two bonds from any carbonyl C. Also included is any aromatic carbon at least three heavy atoms. Additional examples are known in the art or otherwise disclosed herein.

Aside from the lipophilic atom examples provided herein or otherwise known in the art, lipophilic atoms may also be identified using SMARTS patterns with the OEChem Toollit (v1.4.2, OpenEye Scientific Software, Sante Fe, N. Mex.). The SMARTS patterns are: [C;!$(C˜[o,O,n,N]);!$(C˜*˜[o,O,n,N]);!$(C˜*˜C═O);$(C*˜S═O);$(C˜*˜P═O)]; [C;!$(c[o,O,n,N]);!$(c*˜[o,O,n,N;iR]);!$(c*[C;R]═O); and !$(c*˜[S;′R]═O);!$(c*P;!R]═O);!$(c@[*;R]!@![O,N])] [Cl,Br,I]. Alternatively, any substructure search algorithm could be used.

Non-limiting examples of basic ionizable atoms include, any nitrogen capable of adopting a formal charge. Basic ionizable atoms are determined by using Ligprep is (v. 20113, Schrödinger, LLC., New York, N.Y.) to expand all tautomeric and protonation states of a molecule using the “-expand_ite” option. Additional examples are known in the art or otherwise disclosed herein.

Aside from the basic ionizable atom examples provided herein or otherwise known in the art, basic ionizable atom may also be identified using SMARTS patterns with the OEChem Toolkit (v1.4.2, OpenEye Scientific Software, Sante Fe, N. Mex.). The SMARTS patterns are: [N+,n+].

Non-limiting examples of hydrogen bond donor atoms include, any oxygen or nitrogen with an attached hydrogen. Additional examples of a hydrogen bond donor atom include hydroxyl, protonated carboxylate, primary amine, secondary amine, the amide nitrogen, imidazole NH, pyrrole NH, pyrrazole NH, triazole NH, piperidine NH, morpholino NH, piperazine NH, indole NH, isoindole NH, purine NH,.

Aside from the basic ionizable atom examples provided herein or otherwise known in the art, basic ionizable atom may also be identified using SMARTS patterns with the OEChem Toolkit (v1.4.2, OpenEye Scientific Software, Sante Fe, N. Mex.). The SMARTS patterns are: [O,N,n;!HO]. Alternatively, any substructure search algorithm could be used.

In one embodiment of the present invention, the model of the present invention comprises all three descriptors of Formula (I), (II), and (III). In another embodiment of the present invention, the model of the present invention comprises the descriptors of Formula (I) and (II). In another embodiment of the present invention, the model of the present invention comprises the descriptors of Formula (I) and (III). In another embodiment of the present invention, the model of the present invention comprises the descriptors of Formula (II) and (III). In another embodiment of the present invention, the model of the present invention comprises only the descriptor of Formula (I). In another embodiment of the present invention, the model of the present invention comprises only the descriptor of Formula (II). In another embodiment of the present invention, the model of the present invention comprises only the descriptor of Formula (III).

The present invention encompasses the application of the method of the present invention to high-throughput in silico methods of screening not just one compound, but any where from about 10, 25, 50, 100, 1000, 1200, 1500, 2000, 5000, 10000, or more compounds to determine the likelihood the compounds will inhibit the potassium ion current activity of the human ether-a-go-go gene (hERG). Such a method may simply entail automating the method by adding a loop function such that each compound is iteratively analyzed to determine if the compound contains one or more of the descriptors described herein. Such automation methods are readily known in the art and within the scope of the invention.

The term “about” as used herein is meant to mean either 1%, 2%, 3%, 4%, 5%, 6%, 7%, 8%, 9%, 10%, 15%, or even 20% higher or lower than the recited value.

### REFERENCES

(1) Sanguinetti, M. C.; Jiang, C.; Curran, M. E.; Keating, M. T. Cell (Cambridge, Mass.) 1995, 81, 299-307.
(2) Trudeau, M. C.; Warmke, J. W.; Ganetzky, B.; Robertson, G. A. Science (Washington, D.C.) 1995, 269, 92-5.
(3) Keating, M. T.; Sanguinetti, M. C. Cell (Cambridge, Mass., United States) 2001, 104, 569-580.
(4) Viskin, S. Lancet FIELD Full Journal Title:Lancet 1999, 354, 1625-33.
(5) Mitcheson, J. S.; Chen, J.; Lin, M.; Culberson, C.; Sanguinetti, M. C. Proceedings of the National Academy of Sciences of the United States of America 2000, 97, 12329-12333.
(6) Witchel, H. J.; Dempsey, C. E.; Sessions, R. B.; Perry, M.; Milnes, J. T.; Hancox, J. C.; Mitcheson, J. S. Molecular Pharmacology 2004, 66, 1201-1212.
(7) Milnes, J. T.; Crociani, O.; Arcangeli, A.; Hancox, J. C.; Witchel, H. J. British Journal of Pharmacology 2003, 139, 887-898.
(8) Femandez, D.; Ghanta, A.; Kauffman, G. W.; Sanguinetti, M. C. Journal of Biological Chemistry 2004, 279, 10120-10127.
(9) Sanchez-Chapula, J. A.; Navarro-Polanco, R. A.; Culberson, C.; Chen, J.; Sanguinetti, M. C. Journal of Biological Chemistry 2002, 277, 23587-23595.
(10) Ferrer-Villada, T.; Navarro-Polanco, R. A.; Rodriguez-Menchaca, A. A.; Benavides-Haro, D. E.; Sanchez-Chapula, J. A. European Journal of Pharmacology 2006, 531, 1-8.
(11) Recanatini, M.; Cavalli, A.; Masetti, M. Novartis Foundation Symposium 2005, 266, 171-185.
(12) Aronov, A. M. Drug Discovery Today 2005, 10, 149-155.
(13) Recanatini, M.; Poluzzi, E.; Masetti, M.; Cavalli, A.; de Ponti, F. Medicinal Research Reviews 2005, 25, 133-166.
(14) Vaz, R. J.; Li, Y.; Rampe, D. Progress in Medicinal Chemistry 2005, 43, 1-18.
(15) Li, Y.; Cianchetta, G.; Vaz, R. J. Methods and Principles in Medicinal Chemistry 2006, 29, 428-443.
(16) Stansfeld, P. J.; Sutcliffe, M. J.; Mitcheson, J. S. Expert Opinion on Drug Metabolism & Toxicology 2006, 2, 81-94.
(17) Sanguinetti, M. C.; Mitcheson, J. S. Trends in Pharmacological Sciences 2005, 26, 119-124.
(18) Aptula, A. O.; Cronin, M. T. D. SAR and QSAR in Environmental Research 2004, 15, 399-411.
(19) Aronov, A. M.; Goldman, B. B. Bioorganic & Medicinal Chemistry 2004, 12, 2307-2315.
(20) Aronov, A. M. Journal of Medicinal Chemistry 2006, 49, 6917-6921.
(21) Bains, W.; Basman, A.; White, C. Progress in Biophysics & Molecular Biology 2004, 86, 205-233.
(22) Becker, O. M.; Dhanoa, D. S.; Marantz, Y.; Chen, D.; Shacham, S.; Cheruku, S.; Heifetz, A.; Mohanty, P.; Fichman, M,; Sharadendu, A.; Nudelman, R.; Kauffman, M.; Noiman, S. Journal of Medicinal Chemistry 2006, 49, 3116-3135.
(23) Cavalli, A.; Poluzzi, E.; De Ponti, F.; Recanatini, M. Journal of Medicinal Chemistry 2002, 45, 3844-3853.
(24) Choe, H.; Nah, K. H.; Lee, S. N.; Lee, H. S.; Lee, H. S.; Jo, S. H.; Leem, C. H.; Jang, Y. J. Biochemical and Biophysical Research Communications 2006, 344, 72-78.
(25) Coi, A.; Massarelli, I.; Murgia, L.; Saraceno, M.; Calderone, V.; Bianucci, A. M. Bioorganic & Medicinal Chemistry 2006, 14, 3153-3159.
(26) Cianchetta, G.; Li, Y.; Kang, J.; Rampe, D.; Fravolini, A.; Cruciani, G.; Vaz, R. J. Bioorganic & Medicinal Chemistry Letters 2005, 15, 3637-3642.
(27) Ekins, S.; Crumb, W. J.; Sarazan, R. D.; Wikel, J. H.; Wrighton, S. A. Journal of Pharmacology and Experimental Therapeutics 2002, 301, 427-434.
(28) Skins, S.; Balakin, K. V.; Savchuk, N.; Ivanenkov, Y. Journal of Medicinal Chemistry 2006, 49, 5059-5071.
(29) Farid, R.; Day, T.; Friesner, R. A.; Pearlstein, R. A. Bioorganic & Medicinal Chemistry 2006, 14, 3160-3173.
(30) Fioravanzo, E.; Cazzolla, N.; Durando, L.; Ferrari, C.; Mabilia, M.; Ombrato, R.; Parenti, M. D. Internet Electronic Journal of Molecular Design 2005, 4, 625-646.
(31) Gepp, M. M.; Hutter, M. C. Bioorganic & Medicinal Chemistry 2006, 14, 5325-5332.
(32) Pearlstein, R. A.; Vaz, R. J.; Kang, J.; Chen, X.-L.; Preobrazhenskaya, M.; Shchekotikhin, A. E.; Korolev, A. M.; Lysenkova, L. N.; Miroshnikova, O. V.; Hendrix, J.; Rampe, D. Bioorganic & Medicinal Chemistry Letters 2003, 13, 1829-1835.
(33) Rajamani, R.; Tounge, B. A.; Li, J.; Reynolds, C. H. Bioorganic & Medicinal Chemistry Letters 2005, 15, 1737-1741.
(34) Song, M.; Clark, M. Journal of Chemical Information and Modeling 2006, 46, 392-400.
(35) Seierstad, M.; Agrafiotis, D. K. Chemical Biology & Drug Design 2006, 67, 284-296.
(36) Jamieson, C.; Moir, E. M.; Rankovic, Z.; Wishart, C. Journal of Medicinal Chemistry 2006, 49, 5029-5046.
(37) Tang, W.; Kang, J.; Wu, X.; Rampe, D.; Wang, L.; Shen, H.; Li, Z.; Dunnington, D.; Garyantes, T. Journal of Biomolecular Screening 2001, 6, 325-331.
(38) Finlayson, K.; Turnbull, L.; January, C. T.; Sharkey, J.; Kelly, J. S. European Journal of Pharmacology 2001, 430, 147-148.
(39) Wood, C.; Williams, C.; Waldron, G. J. Drug Discovery Today 2004, 9, 434-441.
(40) Gao, F.; Johnson, D. L.; Ekins, S.; Janiszewski, J.; Kelly, K. G.; Meyer, R. D.; West, M. Journal of Biomolecular Screening 2002, 7, 373-382.
(41) Kopman, A. F.; Klewicka, M. M.; Neuman, G. G. Anesthesia & Analgesia (Baltimore) 2000 90, 1191-1197.
(42) Anesth Analg 2000, 91, 67.
(43) Yoo, S.-E.; Cha, O. J. Bulletin of the Korean Chemical Society 1995, 16, 110-12.
(44) Omega; 2.1 ed.; OpenEye Scientific Software, Inc.: Santa Fe, N. Mex., 2006.
(45) MacroModel; 9.1 ed.; Schrodinger, Inc.: New York, N.Y., 2006.
(46) Cruciani, G.; Pastor, M.; Guba, W. European Journal of Pharmaceutical Sciences 2000, 11, S29-S39.
(47) Cruciani, G.; Crivori, P.; Carrupt, P. A.; Testa, B. Theochem 2000, 503, 17-30.
(48) Crivori, P.; Cruciani, G.; Carrupt, P.-A.; Testa, B. Journal of Medicinal Chemistry 2000, 43, 2204-2216.
(49) Volsurf; 4.1.4 ed.; Molecular Discovery, Ltd., 2004.
(50) Hawkins, G. D.; Liotard, D. A,; Cramer, C. J.; Truhlar, D. C. Journal of Organic Chemistry 1998, 63, 4305-4313.
(51) ACD/LogD; 4.76 ed.; Advanced Chemistry Development, Inc.: Toronto, ON, Canada, 2001.
(52) Jakalian, A.; Jack, D. B.; Bayly, C. I. Journal of Computational Chemistry 2002, 23, 1623-1641.
(53) QUACPAC; 1.1 ed.; OpenEye Scientific Software, Inc.: Santa Fe, N. Mex., 2004.
(54) Sutter, J. M., personal communication.
(55) Massart, D. L.; Kaufinan, L.; Rousseeuw, P. J.; Leroy, A. Analytica Chimica Acta 1986, 187, 171-9.
(56) Sinnokrot, M. O.; Sherrill, C. D. Journal of Physical Chemistry A 2003, 107, 8377-8379.
(57) Ringer, A. L.; Sinnokrot, M. O.; Lively, R. P.; Sherrill, C. D. Chemistry—A European Journal 2006, 12, 3821-3828.
(58) Cockroft, S. L.; Hunter, C. A.; Lawson, K. R.; Perkins, J.; Urch, C. J. Journal of the American Chemical Society 2005, 127, 8594-8595.
(59) Lee, E. C.; Hong, B. H.; Lee, J. Y.; Kim, J. C.; Kim, D.; Kim, Y.; Tarakeshwar, P.; Kim, K. S. Journal of the American Chemical Society 2005, 127, 4530-4537.
(60) Perry, M.; Stansfeld, P. J.; Leaney, J.; Wood, C.; de Groot, M. J.; Leishman, D.; Sutcliffe, M. J.; Mitcheson, J. S. Molecular Pharmacology 2006, 69, 509-519.
(61) Roux, B.; MacKinnon, R. Science (Washington, D.C.) 1999, 285, 100-102.
(62) Abraham, M. H. Chemical Society Reviews 1993, 22, 73-83.
(63) FingerprintToolkit; 4.62 ed.; Daylight Chemical Information Systems, Inc.: Santa Fe, N. Mex.
(64) Stouch, T. R.; Kenyon, J. R.; Johnson, S. R.; Chen, X.-Q.; Doweyko, A.; Li, Y. Journal of Computer-Aided Molecular Design 2003, 17, 83-92.

### EXAMPLES

### DESCRIPTION OF THE PREFERRED EMBODIMENTS

### Example 1

### hERG Assay Materials and Methods

Human embryonic kidney (HEK293) cells were stably transfected with human Ether-a-go-go Related Gene (hERG) cDNA for use in the hERG assay. The biophysical and pharmacological properties of recombinant hERG channels expressed in HEK293 cells and of native I_{Kr} channels in human cardiac cells are nearly identical. Several known hERG blockers, including dofetilide, terfenadine, cisapride and E-4031, inhibit recombinant hERG currents in this hERG stable cell line and I_{Kr} current in cardiac myocytes with the same potency.

Membrane current recordings were made with an Axopatch 200 series integrating patch-clamp amplifier (Axon Instruments, Foster City, Calif.) using the whole-cell variant of the patch-clamp technique. For hERG current recording the bath solution, which replaced the cell culture media during experiments, contained: 140 mM NaCl, 4 mM KCl, 1.8 mM CaCl₂, 1 mM MgCl₂, 10 mM glucose, 10 mM HEPES (pH 7.4, NaOH). Borosilicate glass pipettes had tip resistances of 2-4 MΩ when filled with an internal solution containing: 130 mM KCl, 1 mM MgCl₂, 1 mM CaCl₂, 5 mM ATP-K₂, 10 mM EGTA, 10 mM HEPES (pH 7.2, KOH).

hERG-expressing cells were placed in a plexiglass bath chamber, mounted on the stage of an inveited microscope, and perfused continuously with bath solution. To determine potency of test agents for inhibiting hERG current, repetitive test pulses (0.05 Hz) were applied from a holding potential of −80 mV to +20 mV for 2 seconds. Tail currents were elicited following the test pulses by stepping the voltage to −65 mV for 3 seconds. After recording the steady state current for 2-5 minutes in the absence of test agent, the bath solution was switched to one containing the lowest concentration of the agent to be used. The peak tail current was monitored until a new steady-state in the presence of test agent was achieved. This was followed by the application of the next higher concentration of the agent to be tested and this was repeated until all concentrations of test agent had been evaluated. Percent inhibition of tail currents was plotted as a function of test agent concentration to quantify hERG channel inhibition. Compound effects were calculated using tail currents because there are no endogenous tail currents in plasmid-transfected control HEK293 cells. Data were sampled at rates at least two times the low pass filter rate. The flow rate was kept constant throughout the experiments (˜1-5 mL/min). All membrane currents were recorded at room temperature (˜25° C.).

### Example 2

### Computational Methods

### Training and Validation Data

The final collection of molecular descriptors and coefficients were derived using a collection 1075 compounds with either IC₅₀s (289 compounds) or percent inhibition at a single concentration (786 compounds). These 1075 compounds were randomly divided into a training set of 925 compounds and test set of 150 compounds for the purposes of model derivation and testing.

In the time since the model was developed, an additional 1679 compounds have been tested in the manual whole cell patch-clamp hERG inhibition assay. These include 324 IC₅₀s and 1355 single-point percent inhibition measurements. This second data set was used as a true forward-looking validation set as there was no hERG inhibition data available for these compounds at the time of model development.

Estimation of IC₅s from Percent Inhibition

As noted above, the data set used in deriving the model of hERG inhibition was composed of both fully-determined IC₅₀s and percent inhibition at a single concentration. To facilitate modeling, the percent inhibition data was transformed into estimated IC₅₀s using the Logit⁴⁰⁻⁴³ function:

$$Est \cdot {IC}_{50} ≡ \frac{100 - \% inh}{\% inh} \times Conc . \qquad ( 1 )$$

where %inh is the percent inhibition measured at the concentration Conc. The Logit function makes the implicit assumption that at zero concentration there is no inhibition, while at some infinite concentration there is complete inhibition of potassium ion flow through the hERG channel. It also assumes a universal slope to the estimated IC₅₀ curve, which is a substantial source of error in estimating the IC₅₀.

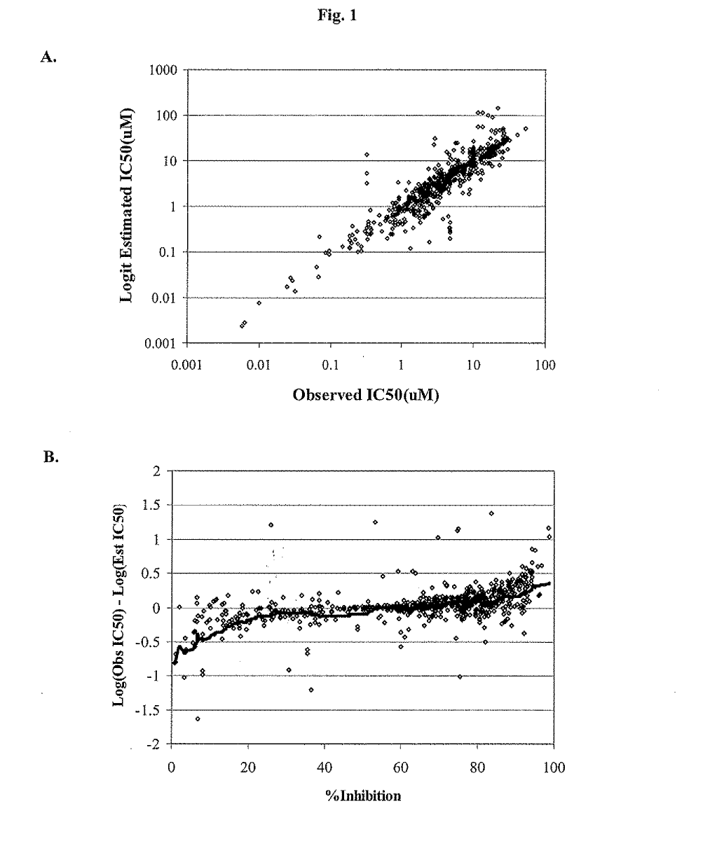

As this estimation of the IC₅₀ for the single-point measurements could introduce significant error, a brief analysis of the transformed data was performed. FIG. 1A shows the relationship between the 613 IC₅₀ measurements available when writing this manuscript and the estimated IC₅₀s that are derived from single-point inhibition data. Overall the relationship is quite good, with a root mean square error (RMSe) of 0.27 log units (μM) and an r²˜0.83. FIG. 1B is a plot of the residuals of the measured IC₅₀ and the estimated IC₅₀ as a function of the percent inhibition at whatever concentration they were determined. A few important observations are drawn from this analysis. First, compounds with percent inhibition between 20% and 80% are well predicted using this approach (median error is 1.3-fold on a μM basis). Second, compounds with very low or very high inhibition were not predicted as well as those compounds having moderate inhibition. The median error of 2.7-fold on a μM basis was observed for compounds having very low or very high inhibition. The maximum error for any transformation was approximately 45-fold.

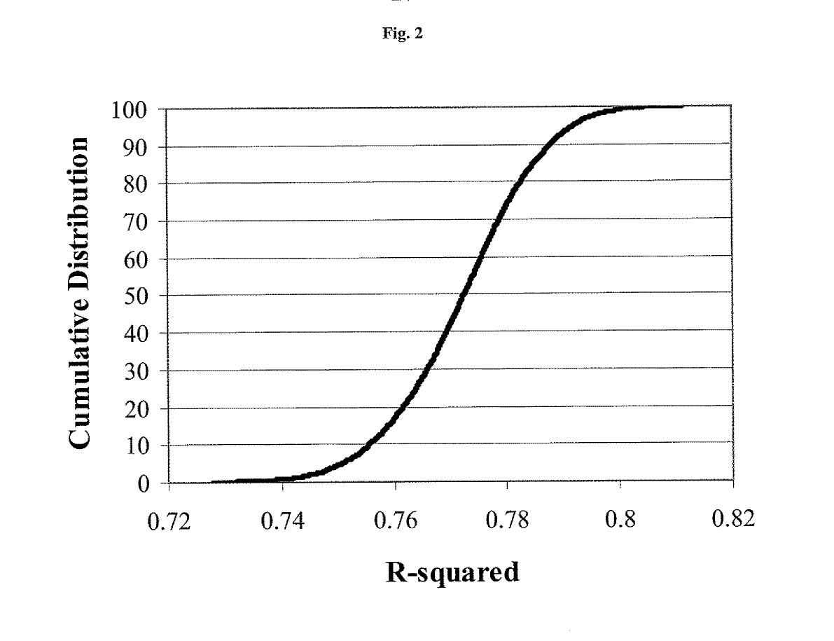

These projected error rates were used in a Monte Carlo simulation to gauge their effect on the quality of the data used to derive the in silico model. The estimated IC₅₀s for the 786 compounds without true IC₅₀s were perturbed by a normal distribution of error, using the moving averages shown in FIG. 1B based on the %inhibition used to estimate the IC₅₀ as dictated above. The correlation coefficient (r²) was then calculated between the original estimated data and the perturbed data. This process was repeated for 5000 iterations, building a distribution of correlations. The cumulative distribution plot shown in FIG. 2 indicates the median r² is approximately 0.77 based on the expected error due to the estimated IC₅₀s. A correlation greater than 0.8 is extremely unlikely and is likely the upper bound on the performance of an in silico model. This estimate is probably overly optimistic as it assumes no error in the measured IC₅₀s and does not account for the peak errors of 45-fold for some single-point to IC₅₀ estimates for the IC₅₀ data.

### Example 3

### Conformation Selection

Initial conformations were generated using Omega⁴⁴ followed by a minimization using Batchmin⁴⁵ using OPLS2005. Conformers within 10 kJ/mol of the minimum energy conformation are retained for use in descriptor generation.

### Descriptor Generation

A multitude of physicochemical descriptors were calculated for consideration as predictors of hERG inhibition. Included among these were Volsurf⁴⁶⁻⁴⁹ descriptors using the Dry and H₂O probes. These descriptors encode hydrophobic and hydrophilic interaction volumes for compounds and have been suggested as useful features in developing predictive ADMET models. Also included were free energies of solvation in various solvents as calculated using the OmniSol program⁵⁰. Finally, the calculated Log D values at pH=6.5 and pH=7.4 were obtained using the ACD/LogD software⁵¹. For each of these features, only the lowest energy conformation generated as described above was utilized.

A number of binary interaction pair descriptors were also calculated. These descriptors are essentially two-point pharmacophore descriptors with through-bond distances. It is expected that they might identify structural groups important for binding along with approximate orientations captured by the bond distances.

Many potential three-dimensional pharmacophores were also generated. For the pharmacophoric descriptors, each of the retained conformers was evaluated for a match. A pharmacophore was considered to be present if it was present in any of the conformations generated above. A pool of pharmacophores was identified for further analysis by searching for pharmacophores present in highly potent blockers (IC₅₀<1 μM) in the training set in a substantially higher proportion than found among the non-potent compounds (IC₅₀>30 μM). Pharmacophores were eliminated from the pool either because of low frequency in the data set as a whole, or were determined to be generally uninteresting in that they contained only lipophilic moieties.

The electrostatic potential around aromatic rings was also utilized as a descriptor. The descriptor was calculated using the lowest energy conformation identified as above. AM1-BCC⁵² charges were calculated using the Molcharge program, a part of the QuACPAC⁵³ suite of tools The compound was placed on a 1 Å grid, and the electrostatic potential was calculated at each grid point. The grid points closer to an aromatic atom than to any non-aromatic atom with an ESP below a threshold of either −1, −5 or −10 kcal/mol are counted. This count is an approximate volume of electrostatic potential proximal to aromatic groups.

### Feature Selection

The current model represents several cycles of hypothesis generation and testing. Consequently, the features present in the model were selected over time rather than in a single optimization method as might be typical for a ligand-based QSAR model. Substantial effort was directed at descriptor selection. Descriptors were selected using either an automated supervised selection algorithm or selected manually based on observed trends in discovery projects.

The supervised descriptor selection algorithm was developed in-house. This algorithm, similar to others in the literature, first removes descriptors with greater than 90% identical values. A complete pairwise correlation matrix is then generated for the entire remaining descriptor pool. Subsets of descriptors are selected with the constraint that no two included descriptors have a pair wise r²>0.9.

The supervised algorithm uses simulated annealing with a leave-10%-out PRESS cross-validation function to evaluate potential models. Initially, a random subset of descriptors is chosen. Following this, a single descriptor is removed and replaced with another feature from the pool. This replacement is done randomly, with the exception that the new feature cannot be correlated with any other feature already present in the model. The PRESS score is then evaluated for this new model. If the score is an improvement over the previous model, the result is accepted and the procedure repeats. If the model is not an improvement, the model may still be accepted based on a probability derived from the Boltzmann distribution.

The algorithm used here fixes the initial temperature such that 80% of the detrimental steps are accepted originally⁵⁴. The temperature is then decreased by 25% every 1000 iterations. The selection process halts when no model is accepted for 900 iterations.

The descriptors selected by the automated routine were each manually analyzed before being included in the final model. For example, descriptors are often highly correlated with other available descriptors. Descriptors that appeared to make more physical sense with respect to existing knowledge and/or literature for hERG inhibition were preferred over correlated descriptors with unclear interpretations.

Several descriptors were selected manually in response to a particular trend seen within a discovery project. A primary example in the model would be the electrostatic potential descriptor. This descriptor was included based on the observation of a 5-fold increase in potency for a 4-fluoro substitution relative to an unsubstituted phenyl, which was then followed by 17-fold increase in potency for the 4-chloro substitution. In contrast, a 2-fold and 3-fold loss in activity was observed for the 4-amino and 4-methyl substitutions, respectively. A search of the internal database indicated that this trend was fairly commonplace.

### Model Training

Once descriptors were selected, they were utilized in a least median squares (LMS) regression.⁵⁵ Descriptors were range scaled so that the minimum value for each descriptor was 0.0 and the maximum value was 1.0. LMS regression is a robust regression method that de-emphasizes leverage points in determining coefficients. The method searches for the set of coefficients that minimizes the median squared residual, rather than the sum of the squared residuals as in ordinary multiple regression. The implementation used here was programmed in our laboratory.

It will be clear that the invention may be practiced otherwise than as particularly described in the foregoing description and examples. Numerous modifications and variations of the present invention are possible in light of the above teachings and, therefore, are within the scope of the appended claims.

The entire disclosure of each document cited (including patents, patent applications, journal articles, abstracts, laboratory manuals, books, or other disclosures) in the Background of the Invention, Detailed Description, and Examples is hereby incorporated herein by reference. Further, the hard copy of the sequence listing submitted herewith and the corresponding computer readable form are both incorporated herein by reference in their entireties.

What is claimed is:

1. A method of predicting the likelihood that a compound will inhibit the potassium ion current activity of the human ether-a-go-go gene (hERG), comprising the step of determining whether said compound comprises one or more of the descriptors selected from the group consisting of: a.) the descriptor according to Formula (I); b.) the descriptor according to Formula (II); and c.) the descriptor according to Formula (III).

2. The method according to claim 1 wherein said method comprises determining whether said compound comprises both the descriptor according to Formula (I) and the descriptor according to Formula (II).

3. The method according to claim 2, wherein a compound comprising both the descriptor according to Formula (I) and the descriptor according to Formula (II) has a lower likelihood of inhibiting the potassium ion current activity of the human ether-a-go-go gene (hERG) relative to a compound having only the descriptor according to Formula (I).

4. The method according to claim 1, wherein said method comprises determining whether said compound comprises the descriptor according to Formula (II).

5. The method according to claim 4, wherein a compound comprising the descriptor according to Formula (III) has a higher likelihood of inhibiting the potassium ion current activity of the human ether-a-go-go gene (hERG) relative to a compound lacking this descriptor.

6. The method according to claim 1, wherein said method comprises determining whether said compound comprises the descriptor according to Formula (I), and further comprises determining whether said compound comprises an aromatic ring with sufficient electrostatic potential to enable Pi-stacking.

7. The method according to claim 6, wherein a compound comprising the descriptor according to Formula (I) in conjunction with an aromatic ring capable of Pi-stacking, has a higher likelihood of inhibiting the potassium ion current activity of the human ether-a-go-go gene (hERG) relative to a compound having only the Formula (I) descriptor.

8. The method according to claim 5, wherein said compound has about 3 fold higher likelihood of inhibiting the potassium ion current activity of the human ether-a-go-go gene (hERG) relative to a compound lacking this descriptor.

9. The method according to claim 6, wherein said method comprises determining whether said compound comprises the descriptor according to Formula (I), and further comprises determining whether said compound comprises an aromatic ring with sufficient electrostatic potential to enable Pi-stacking wherein said aromatic ring is substituted with at least one electron withdrawing group.

10. The method according to claim 9, wherein said compound has about a 5 to about a 30 fold higher likelihood of inhibiting the potassium ion current activity of the human ether-a-go-go gene (hERG) relative to a compound containing an unsubstituted aromatic ring.

11. The method according to claim 6, wherein method said comprises determining whether said compound comprises the descriptor according to Formula (I), and further comprises determining whether said compound comprises an aromatic ring with sufficient electrostatic potential to enable Pi-stacking wherein said aromatic ring is substituted with at least one electron donating group.

12. The method according to claim 11, wherein said compound has about a 2 to about a 5 fold lower likelihood of inhibiting the potassium ion current activity of the human ether-a-go-go gene (hERG) relative to a compound containing an unsubstituted aromatic ring.
